(19) United States
(12) Patent Application Publication
(10) Pub. No.: US 2002/0022017 A1
(43) Pub. Date: Feb. 21, 2002

(54) Regulation of systemic immune responses utilizing soluble CD40

(76) Inventor: Hua Yu, Tampa, FL (US)

Correspondence Address: PEPPER, HAMILTON LLP, 600 Fourteenth Street, N.W., Washington, DC, 20005-2004, US

(21) Appl. No.: 09/832,865

(22) Filed: Apr. 12, 2001

Related U.S. Application Data
(60) Provisional application No. 60/196,489, filed on Apr. 12, 2000.

(51) Int. Cl. A61K 48/00

(52) U.S. Cl. 424/93.21; 424/85.1

(57) ABSTRACT

A method of altering the specific, systemic immune response of an individual to an endogenous tumor or specific antigen by the administration, directly or as a gene-product, of a soluble form of CD40, optionally in combination with a cytokine and/or cell-based or isolated antigen. The target antigen may be a tumor cell, a tumor cell antigen, or other antigen to which a systemic immune response is desirable. Co-administration of a soluble form of CD40 and GM-CSF provides effective immunotherapy directed towards the treatment and/or prevention of otherwise poorly immunogenic tumors.

### CROSS REFERENCE TO RELATED APPLICATIONS

The present application claims priority to U.S. Provisional Application Ser. No. 60/196,489, filed Apr. 12, 2000, the disclosure of which is incorporated by reference herein.

### FIELD OF THE INVENTION

The present invention pertains generally to methods for treating or preventing animal disease, including human disease (e.g., cancer or other neoplastic diseases), using therapeutic agents or vaccines. These therapeutic agents or vaccines comprise a soluble form of CD40 (sCD40) or can cause sCD40 to be expressed in vivo. More specifically the invention pertains to methods for delivering an sCD40, either alone or in combination with cytokines, autologous or heterologous cells, antigens, or other compounds, substances, cells or tissues, to evince a systemic immune response in the treatment or prevention of a pathological or potentially pathological condition, such as tumor cells or tissue, or a cancerous phenotype. The present invention also relates to cell lines and compositions capable of providing sCD40 to a patient suffering from a condition responsive to the administration of sCD40, either alone or in one or more of the above-mentioned combinations. Such a patient might be suffering from cancer, for example.

### BACKGROUND OF THE INVENTION

Immunotherapy is a promising therapeutic approach for the treatment of cancer and is based on the premise that the failure of the immune system to reject spontaneously arising tumors is related to the failure of the immune system to appropriately respond to tumor antigens. In a functioning immune system, tumor antigens are processed and expressed on the cell surface in the context of major histocompatability complex (MHC) class I and II molecules, which are in humans also termed “human leukocyte associated” (HLA) molecules. Complexes of MHC class I and II molecules with antigenic peptides are recognized by CD8+ and CD4+ T cells, respectively. This recognition generates a set of secondary cellular signals and the paracrine release of specific cytokines or soluble so-called “biological response mediators,” which mediate interactions between cells and stimulate host defenses to fight off disease. The release of cytokines then results in the proliferation of antigen-specific T cells.

Immunotherapy involves the injection of tumor cells to generate either a novel or an enhanced systemic immune response. The ability of this immunotherapeutic approach to augment a systemic T cell response against a tumor has been previously disclosed, e.g., amongst others, see International Application WO 92/05262; Fearon et al., Cell, 60, 397-403 (1990); Dranoff et al., Proc Natl. Acad. Sci., 90, 3539-43 (1993); U.S. Pat. No. 6,187,306 B1, to Pardoll et al., issued Feb. 13, 2001; and U.S. Pat. No. 5,904,920 to Dranoff et al., issued May 18, 1999. The injected tumor cells are usually altered to enhance their immunogenicity, such as by admixture with non-specific adjuvants, or by genetic modification of the cells to express cytokines or other immune co-stimulatory molecules. Cytokines and combinations of cytokines have been shown to play an important role in the stimulation of the immune system. For example, U.S. Pat. No. 5,098,702 describes the use of combinations of TNF, IL-2 and IFN-β in synergistically effective amounts to combat existing tumors. U.S. Pat. No. 5,078,996 describes the activation of macrophage nonspecific tumoricidal activity by injecting recombinant GM-CSF to treat patients with tumors.

Tumor cells used in immunotherapy can be autologous, i.e., derived from the same host as is being treated, or the tumor cells can be MHC-matched, or having the same, or at least some of the same, MHC complex molecules.

Cytokine-mediated immunotherapy shows promise for cancer treatment. In particular, GM-CSF gene-based cancer vaccines can elicit potent protective immunity against tumor challenge. However, in hosts with established tumors, vaccines such as GM-CSF/IL-12, for example, produce little inhibitory effect against the growth of the tumors.

CD40 is a membrane differentiation antigen expressed on all antigen-presenting cell types, including B cells, dendritic cells and macrophages. CD40 ligand (CD40L) is predominantly expressed on activated CD4+ T cells. The interaction of CD40 and its ligand CD154 (CD40L) plays an important role in the induction of cellular immune responses. Expression of CD40L on CD8+ T cells and natural killer (NK) cells has been described, for example, by Ridge et al., A conditioned dendritic cell can be a temporal bridge between a CD4⁺T-helper and a T-killer cell, Nature 393:474-477 (1998), and Gurunathan et al., CD40 ligand/trimer DNA enhances both humoral and cellular immune responses and induces protective immunity to infectious and tumor challenge, J. Immunol. 161:4563-4571 (1998). Signaling through CD40 by CD40L is important for APC (antigen presenting cell) function and is critical for T cell activation in vitro. Studies in CD40 knockout mice show that the absence of co-stimulation of T cells through CD40 ligand inhibits development of helper function. Alternatively, for example, over-expression of the membrane-bound CD40 (mCD40) in murine tumor cell line P815 stimulates T cell activation in vitro and increases the immunogenicity of tumor cells in vivo. However, no therapeutic anti-tumor effect of mCD40 has been reported. Activating CD40 on APC results in T cell and NK cell-mediated anti-tumor effects. However, the potential of engaging CD 40L on T cells for tumor immunotherapy has not been evaluated.

Therefore there is a need within the field of immunotherapy for improved vaccines that have improved effectiveness against established tumors. In particular, immunotherapeutic agents that evoke an improved systemic immune response upon challenge would be useful in the treatment and prevention of disease, such as cancer. The present invention addresses these needs and more by providing immunotherapeutic compositions and methods of their use based on a soluble form of CD40. These advantages and more will be apparent to one of skill in the art upon consideration of the present disclosure.

### SUMMARY OF THE INVENTION

The present invention is based upon the determination that tumor cells expressing a soluble form of CD40, optionally in combination with certain cytokines, can confer long term specific systemic immunity to individuals receiving such cells. This determination, and developments therefrom, provide for the regulation, either in a stimulatory or suppressive way, of the immune response of a subject.

The present invention is useful in both preventative and therapeutic applications. Thus, the present invention will find application, for example, in protecting a patient from the progression of a tumor, bacterial, or viral infection such as AIDS, transplanted tissue rejection, or autoimmune response. The application of the present invention can be adapted to the treatment or prevention of particular diseases through the choice of antigen optionally co-administered, and through the choice of cytokine or cytokines also optionally co-administered. These optional components are co-administered to a subject with a soluble form of a CD40. The methods of the present invention can be effected using a number of techniques including, but not limited to, cell therapy, gene therapy and/or protein therapy.

In one aspect of the present invention, there is disclosed a method for regulating the immune response of an individual to a target antigen. The regulation is achieved by administering to the individual the target antigen under conditions whereby a soluble form of CD40 is also delivered to the immune system of the individual. A systemic immune response to the specific antigen is thus induced in the individual.

Another aspect of the invention utilizes cells, for example, tumor cells, from an individual to provide the antigen. For example, a tumor cell is “engineered” to provide a soluble form of CD40 when reintroduced into an individual. Specific embodiments of this aspect of the invention utilize tumor cells that are provided with a transgene that produces sCD40. Preferably, the tumor cells are rendered proliferation incompetent prior to administration, for example by irradiation. Cells can be administered by any effective fashion including, but not limited to, systemically, peritoneally, intramuscularly, or intradermally.

In a particular embodiment of the invention, a means for supplying a cytokine is provided in addition to a means for supplying a sCD40. The substances of the present invention can be supplied by injection or can be introduced in a programmed fashion using commercially available pumps. Specific embodiments provide for means whereby IL-12 and/or GM-CSF are simultaneously or sequentially provided to the individual. In certain embodiments, the cytokine and sCD40 are provided as a fusion protein, for example, via expression of an appropriate transgene.

It is a further object of the present invention to facilitate the use of sCD40 as a cancer immunotherapeutic, for example, via combination with cytokine gene-based cancer cell vaccines.

In a particular embodiments of the invention, co-administration of an antigen is not required. For example, a soluble form of CD40 can be administered to an individual alone or in combination with one or more cytokines, in order to induce a systemic immune response to, for example, a pre-existent antigen, such as a poorly immunogenic tumor.

In further embodiments of the invention, sCD40 may be provided as a purified protein, optionally in combination with an antigen, which may also be in a purified state. Further specific embodiments provide for co-administration of a cytokine, also optionally provided in a purified state. In such embodiments, the administration of sCD40 is consistent with its use as an immune adjuvant for antigen-based immune therapies.

To clarify certain aspects of the present invention, while expression of the membrane-bound CD40 in vivo results in only slight, if any, anti-tumor responses, expression of a soluble form of CD40 protein (sCD40), preferably but not necessarily at the tumor site, results in therapeutic anti-tumor effects in poorly immunogenic tumor models, such as for example B16 melanoma. Importantly, while GM-CSF-cDNA-based cancer vaccine, one of the most potent cancer vaccines, has little effect on the growth of tumors in several established murine tumor models, local immunization with irradiated tumor cells expressing the sCD40 in combination with GM-CSF induces potent growth inhibition of distant established murine tumors. In vivo expression of the sCD40 also stimulates Th1 cytokine production, stimulating cytolytic activities of both cytotoxic T lymphocytes and natural killer cells in murine tumor models but not in control CD40L-/-mice. Thus, a sCD40 protein can induce potent anti-tumor T and NK cell activities in vivo, and amplify dramatically the anti-tumor effects of GM-CSF based cancer vaccines.

Therefore, as shown herein, in contradistinction to membrane bound CD40 which is relatively ineffective at stimulating anti-tumor immunogenicity in vivo, transgenic expression of sCD40 in vivo results in a therapeutic anti-tumor effect in a variety of tumor models including B16 melanoma. Furthermore, sCD40 transgenic expression is able to enhance the therapeutic efficacy of GM-CSF-based cancer vaccine. Hence, in addition to its own anti-tumor activity, the present invention has the capacity to improve on the anti-tumor effect of existing cancer vaccines and, presumably, any cancer vaccines that are similar to existing one, which are developed in the near future.

### BRIEF DESCRIPTION OF THE FIGURES

FIG. 1 illustrates a soluble CD40 expression vector.

FIG. 2 illustrates a soluble CD40/GM-CSF fusion protein vector.

FIG. 3 illustrates natural killer cell stimulation.

FIG. 4 illustrates cytotoxic cell (CTL) assays.

FIG. 5 illustrates cytokine production.

FIG. 6 illustrates a soluble CD40 therapeutic vaccine.

FIG. 7 illustrates soluble CD40 cancer gene therapy.

FIG. 8 illustrates vaccine therapy, J558 myeloma tumors.

FIG. 9 illustrates vaccine therapy, MOPC myeloma tumors.

FIG. 10 illustrate vaccine therapy, Meth-A sarcoma tumors.

FIG. 11 illustrates soluble CD40GM-CSF fusion protein plasmid vaccine gene therapy.

FIG. 12 illustrates detection of CD40 and GM-CSF proteins in transfected 3T3 fibroblasts.

### DETAILED DESCRIPTION OF PREFERRED EMBODIMENTS

The term “systemic immune response” as used herein is meant to be an immune response that is not localized, but affects the subject as a whole, whereby specific subsequent responses to the same stimulus can be elicited.

The term “subject” is used herein to refer to an individual mammal, including humans.

The term “therapeutically effective amount” as used herein refers to that amount of a substance that, by administration in one or more dosages, elicits a systemic immune response in a subject. It is understood that such an amount may differ from individual to individual and also by the mode of administration or other specific details, such as the specific cytokine, specific tumor cell, or nature of the composition administered. The appropriate amount may be determined by any of several well-established methodologies, such as determination of the maximal tolerable dose of bioactive substance per kg weight. Extrapolation from such determinations are regularly made by those of skill in the art to obtain sub-toxic yet effective dosages. In particular combinations, sCD40 may be combined with another active substance in molar ratios ranging from about 0.1:1.0 to about 10.0:1.0, preferably between about 0.5:1.0 to about 5.0:1.0, more preferably 1.0:1.0.

Cells are “proliferation incompetent” if they are unable to divide subsequent to administration to a subject. A preferred method for rendering cells proliferation incompetent is by gamma irradiation. Typically, a minimum dosage of 3500 rads is acceptable, but dosages up to 30,000 rads may be used. It is preferred that the cells retain the ability to express such cytokines, fusion proteins, or other recombinant products for which the cells may contain specific nucleic acids. Other means for rendering cells proliferation incompetent include treatment with mitomycin C and functionally related compounds.

In certain embodiments, cells are “genetically engineered” to express sCD40, cytokines, or fusion proteins thereof. It is understood that the term “genetically engineered” encompasses all means known in the art to direct expression of a recombinant protein, including but not limited to plasmids, retroviral vectors, and any vector that is suitable for introducing a nucleic acid into a eukaryotic cell. The term “recombinant” merely denotes the linkage of nucleic acid sequences that are not typically joined as isolated from natural sources. In the recombinant vectors of the present invention, preferably all signals typically used to direct transcription, mRNA splicing, translation, and post-translational modification of the desired recombinant product are present sufficient to produce a biologically active product within the terms of the present invention. Such signals may also include a promoter, which may be a tissue-specific promoter that preferably directs expression within a specific tissue, or a tumor-specific promoter such as the carcino-embyogenic antigen for colon carcinoma (see Schrewe et al., Mol. Cell. Biol., 10, 2738-2748 (1990)). Suitable promoters can also include the protein's own promoter, a constitutive promoter such as, for example, cytomegalovirus immediate early promoter/enhancer, type 5 major late promoter, the Rous sarcoma virus long terminal repeat, and others known in the art. It is understood that optimization of expression for a particular recombinant protein is routinely performed.

The term “fusion protein” denotes the covalent attachment of two or more proteins whereby at least one biological activity of each protein is retained when the fusion protein is expressed. Thus, in a preferred embodiment, sCD40 is expressed as a fusion protein with GM-CSF, in which a linker polypeptide is encoded within the vector in such a manner as to encode an in-frame polypeptide that connects the two proteins. A suitable linker polypeptide is that encoded by the sequence 5′-GCCGCCGCCGCC-3′.

By the term “suppressing the growth of a tumor” herein is meant the suppression, regression, partial or complete disappearance of a preexisting tumor. The definition encompasses any diminution in the size, potency, growth rate, appearance or feel of a preexisting tumor. Notwithstanding the above, this term also meant to encompass a non-progressing status, i.e., the tumor stays the same but does not necessarily regress.

The term “co-administering” means that the two compounds referred to are both provided to the immune system of a subject. The term does not denote a specific order of administration, and neither does it imply whether or not the two compounds are first combined prior to administration.

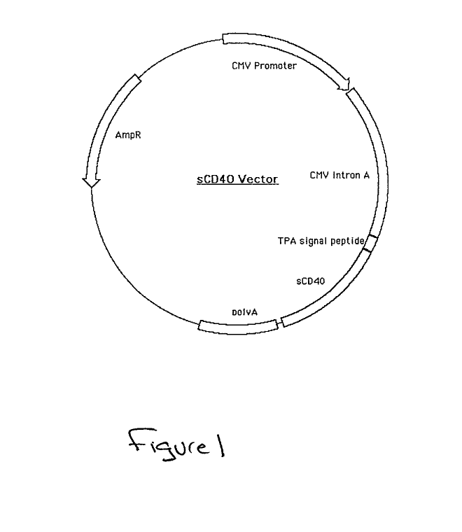

Referring now to the figures, FIG. 1 illustrates a soluble CD40 expression vector. A CD40 gene lacking the transmembrane domain is cloned into a suitable expression vector, preferably comprising a suitable promoter (e.g., CMV promoter) and a secretion-directing signal peptide (e.g., TPA signal peptide).

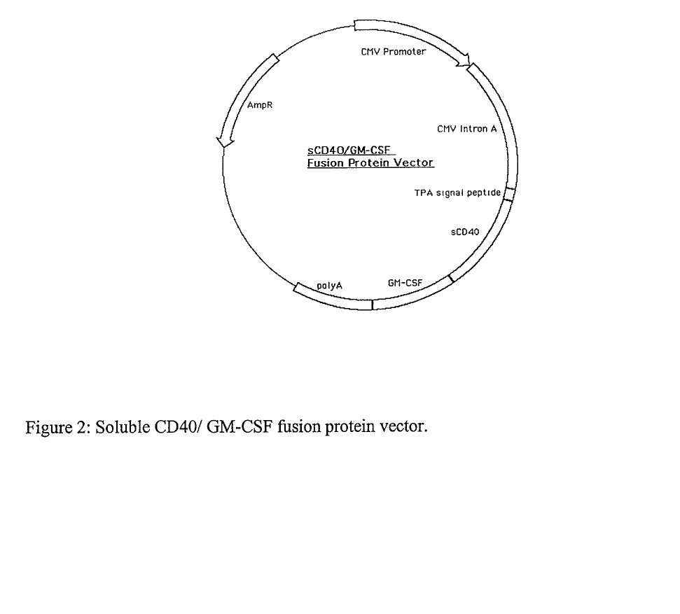

In FIG. 2 a soluble CD40/GM-CSF fusion protein vector is illustrated. In this particular embodiment, the genes for sCD40 and GM-CSF are linked by an in-frame linker region.

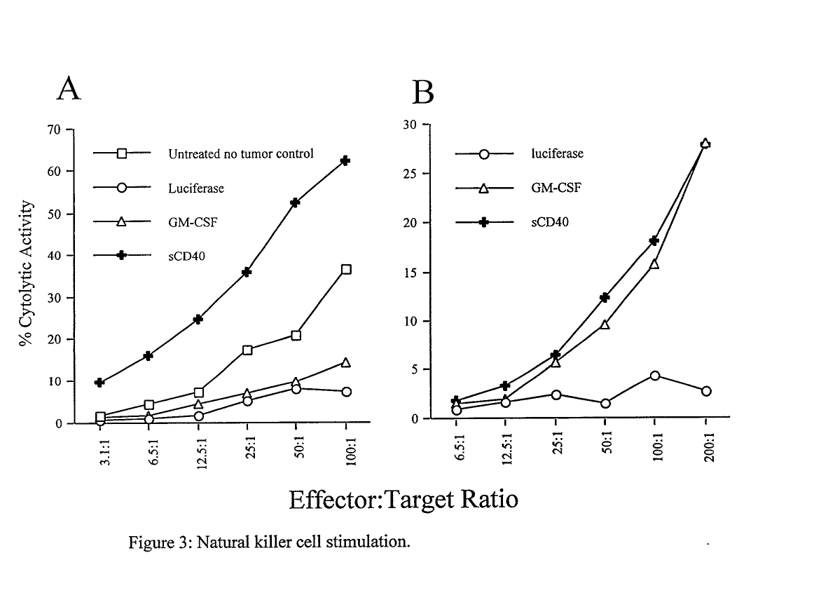

FIG. 3 illustrates natural killer cell stimulation. A, MPC11 myeloma tumor bearing Balb/C mice are treated with three injections of 2 micrograms plasmid DNA encoding sCD40 (plus); GM-CSF (triangle); or luciferase (open circle) injected intra-muscular (bilaterally) and intra-tumorally using a cationic lipid transfection reagent. Plasmid DNA is injected days 4 and 8 post tumor challenge, NK assay is performed on day 11. NK activity is measured by chromium-51 release assay using YAC-1 cells as targets. Tumors are between 9.5 and 2.5 mm diameter at the time of NK assay. B, Naïve mice are transfected via gene gun to the skin with 12 μg plasmid DNA, NK assay performed 3 days post transfection.

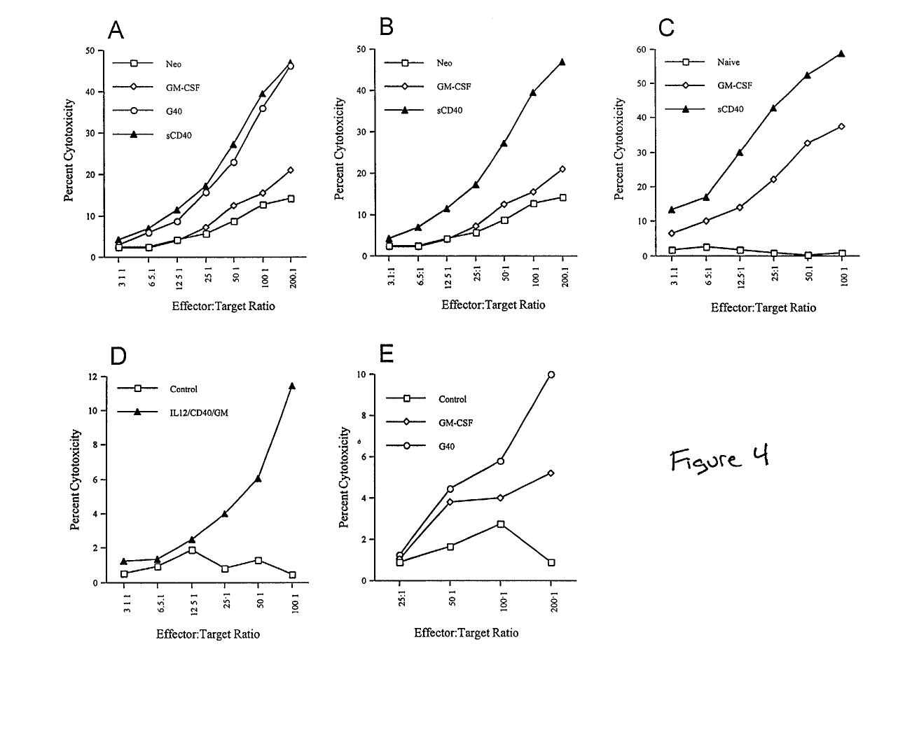

In FIG. 4 cytotoxic cell (CTL) assays are illustrated. A and B, syngenic mouse fibroblast cell lines are transfected with plasmid DNA encoding sCD40 (triangle); GM-CSF (diamond, ⋄); G40(circle, ∘); or Neo (square, □) and selected with G418. Stably transfected fibroblasts are then mixed with MPC11 tumor cells (1:1 ratio), irradiated, and used as a vaccine. Mice are vaccinated twice at one week intervals and CTL activity measured via chromium release assay 21 days after the first vaccine. C, MPCL11 myeloma cells are gene gun transfected with plasmid DNA encoding sCD40 (triangle); SM-CSF (diamond, ⋄) or control (square,□), irradiated, and injected into mice and assayed as above. D, Balb/C mice are challenged with J558 myeloma cells and treated with therapeutic fibroblast/J558 cell vaccine. Therapeutic vaccines, consisting of gene gun transfected irradiated mouse fibroblast cells expressing IL12/sCD40/GM-CSF mixed with J558 cells (1:1), are given on days 5 and 12 post tumor challenge. Day 35 post tumor challenge, surviving mice (40%) are sacrificed and CTL activity measured using chromium release assay. Control mice are naive age matched controls. E, C57/6 mice challenged with B16 melanoma tumor and treated via direct tumor injection of plasmid DNA encoding GM-CSF (diamond, ⋄); G40 (circle, ∘) or control (square, □) (10 micrograms) and cationic lipid transfection reagent 4 and 8 days post transfection. Mice are sacrificed at 14 days and CTL activity is measured by chromium release assay.

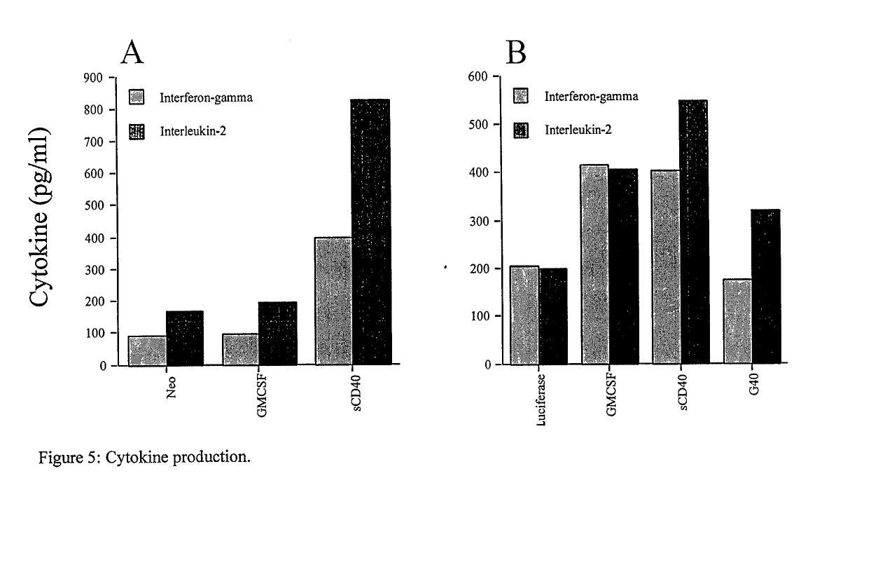

FIG. 5 illustrates cytokine production. A, supernatants from Cytotoxic T cell (CTL) assay are assayed for interferon-gamma and interleukin-2. Syngenic mouse fibroblast cell lines are transfected with plasmid DNA encoding sCD40; GM-CSF; or Neo and selected with G418. Stably transfected fibroblasts are then mixed with MPC11tumor cells (1:1ratio), irradiated and used as a vaccine. Mice are vaccinated twice at one week intervals, and spleens removed 21 days after first vaccine. Splenocyte cultures are treated with ACK to remove RBC, and placed in a 24 well plate at a concentration of 5×10⁶ splenocytes per well with 5×10⁴ irradiated target cells (MPC11). Culture supernatants are assayed after 24 hours. B, Supernatants from natural killer cell assay are assayed for interferon-gamma and interleukin-2. Naive mice are transfected via gene-gun to the skin with 12 micrograms plasmid DNA encoding sCD40; GM-CSF; luciferase; or Neo. Three days post transfection spleens are removed, stomachered, treated with ACK to remove RBC, and placed in a 24 well plate at a concentration of 5×10⁶ splenocytes per well with 5×10⁴ irradiated target cells (YAC-1). After 48 hours incubation at 37C and in the presence of anti-CD3, supernatants are taken and assayed via ELISA for interferon gamma and IL-2.

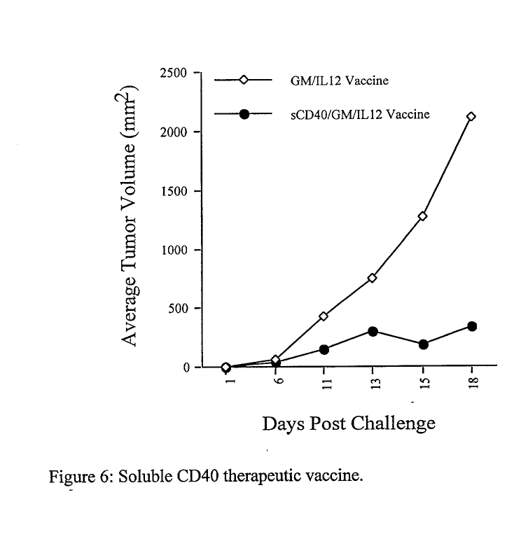

FIG. 6 illustrates a soluble CD40 therapeutic vaccine. C57/6 mice are challenged with 10⁵ B16 melanoma cells injected intradermally on shaved abdomen. Therapeutic vaccines consisting of gene gun transfected irradiated B16 cells are given on days 7 and 12 post tumor challenge. The first vaccine (day 7) is with B16 cells transfected with plasmid DNA encoding soluble CD40 and GM-CSF (open circle) or GM-CSF alone (diamond), the second vaccine (day 12) is with soluble CD40 and IL-12 (open circle) or IL12 alone (diamond). Each vaccination is intradermal on the abdomen bilaterally, with approximately 1.5×10⁶ cells per injection.

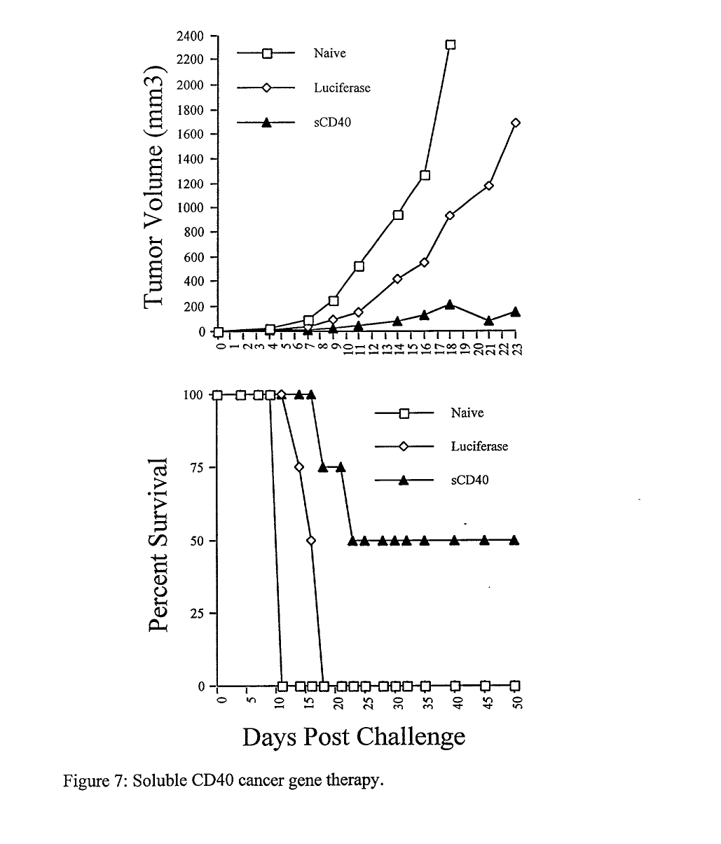

In FIG. 7 soluble CD40 cancer gene therapy is illustrated. C57/6 mice are challenged with 10⁵ B16 melanoma cells injected intradermally on the abdomen. Gene therapy treatments consist of three injections of 2 micrograms of plasmid DNA expressing sCD40 (triangle); luciferase (diamond) or control (square) injected intra-muscular(2) and intratumorally (1) using cationic lipid transfection reagent. Plasmid DNA is injected days 4 and 8 post tumor challenge. Tumors are between 1.5 and 4.2 millimeter in diameter at the time of first treatment.

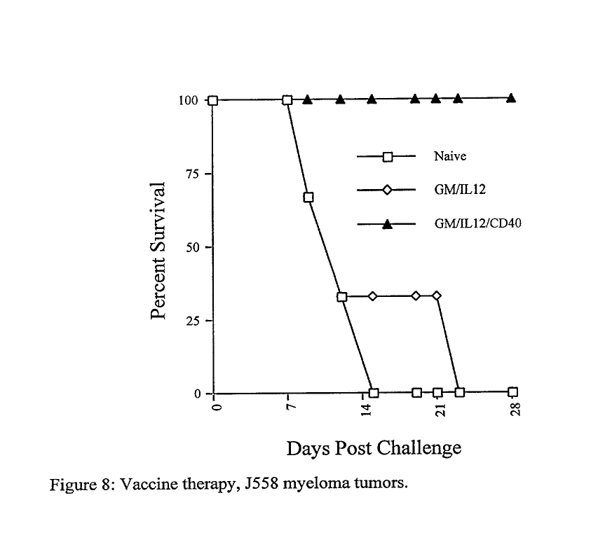

In FIG. 8 vaccine therapy is illustrated, J558 myeloma tumors. Balb/C mice are challenged with 10⁶ J558myeloma cells injected intradermally on the abdomen. Therapeutic vaccine consisting of gene gun transfected irradiated mouse fibroblast cells mixed with J558 cells (1:1), are given on days 7 and 13 post tumor challenge. The first vaccine (day 7) is with fibroblast cells transfected with plasmid DNA expressing soluble CD40 and GM-CSF (triangle); GM-CSF alone (diamond) or control (square), the second vaccine (day 13) is with soluble CD40 and IL-12 (triangle; IL12 (diamond) or control (square). Each vaccination is intradermal on the abdomen bilaterally, with approximately 2×10⁶ cells per injection.

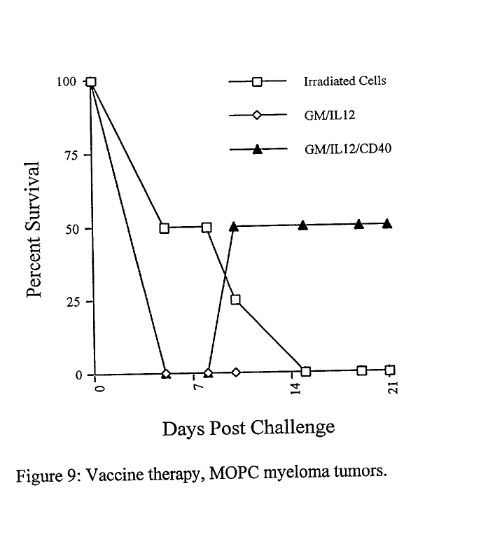

FIG. 9 illustrates vaccine therapy, MOPC myeloma tumors. Balb/C mice are shaved (abdomen) and challenged with 10⁶ MOPC myeloma cells. Vaccine consists of 2 intradermal injections of irradiation tumor cells transfected via gene gun with plasmid DNA (2 micrograms). Mice are vaccinated 5 days post tumor challenge, with vaccine expressing sCD40 and GMOCSF (triangle); GM-CSF (diamond) or control (square), and 12 days post tumor challenge with vaccine expressing sCD40 and IL12 (triangle); IL12 (diamond) or control (square). Tumors are greater than 2 mm in diameter at the time of first vaccination.

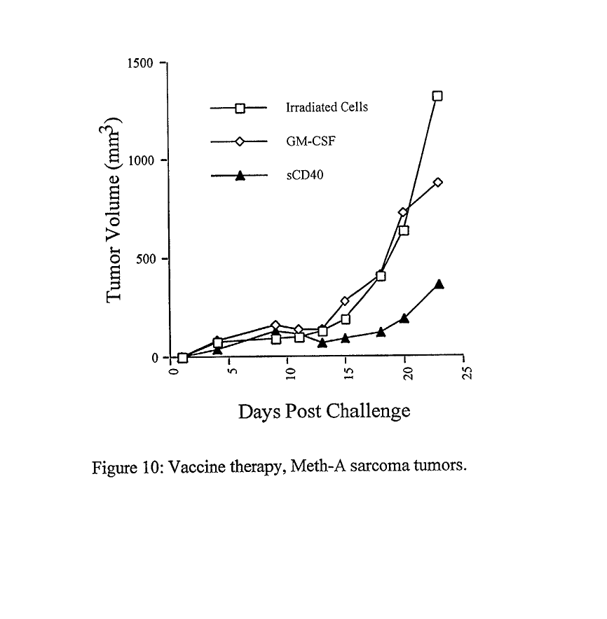

FIG. 10 illustrates vaccine therapy, Meth-A sarcoma tumors. Balb/C mice are shaved (abdomen) and challenged with 10⁶ Meth-A sarcoma cells. Vaccine consists of 2 intradermal injections of irradiated tumor cells transfected via gene gun with plasmid DNA (2 micrograms). Mice are vaccinated 5 and 9 days post tumor challenge, with vaccine expressing sCD40 (triangle); GM-CSF (diamond); or control (square) tumors are greater than 5 mm in diameter at the time of the first vaccination.

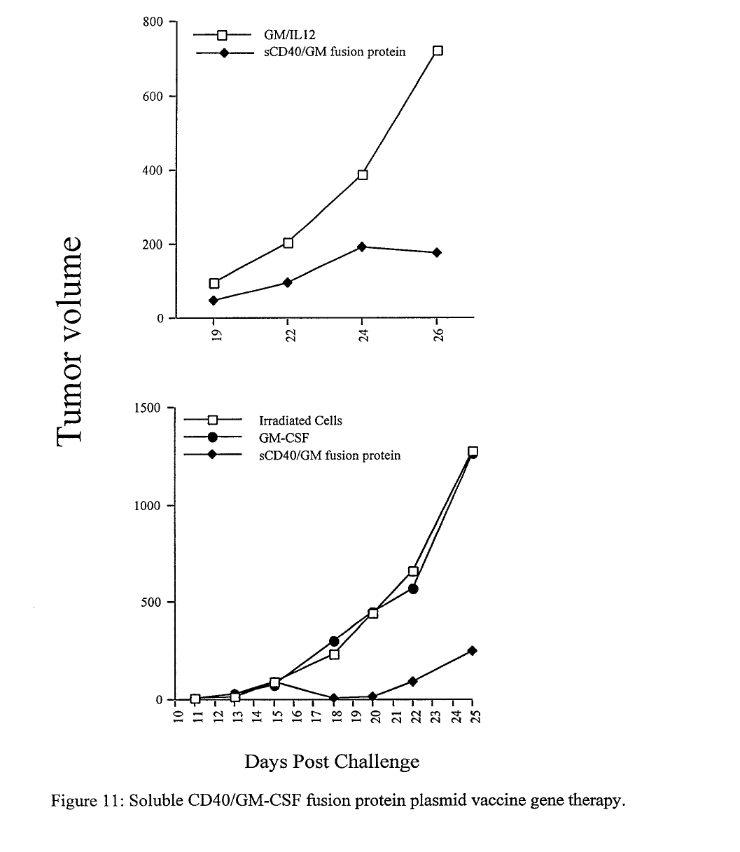

FIG. 11 illustrates soluble CD40/GM-CSF fusion protein plasmid vaccine gene therapy. C57/6 mice are challenged with 10⁶ B16 melanoma cells injected intradermally on the abdomen. A. Therapeutic vaccines consist of either irradiated B16 cells or gene gun transfected irradiated B16 cells expressing sCD40-GM-CSF fusion protein (diamond) or GM-CSF-IL12 fusion protein (square) injected on days 5 and 11 post tumor challenge. B.B16 cells are transfected with plasmid DNA expressing either sCD40/GM-CSF fusion protein (diamond) or GM-CSF (filled circle) or control (square). Each vaccination is intradermal on the abdomen bilaterally, with approximately 1.5×10⁶ cells per injection.

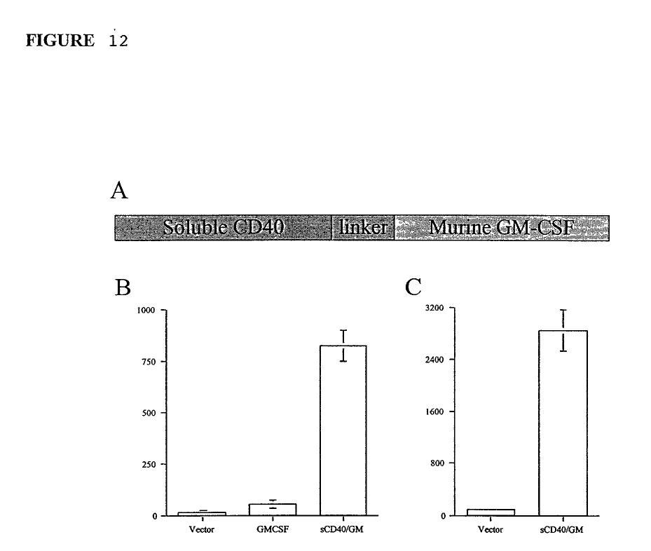

In FIG. 12 is illustrated the detection of both CD40 and GM-CSF proteins in 3T3 fibroblasts transfected with a construct encoding the fusion protein of GM-CSF and sCD40. A. sCD40/GM-CSF construct. B. In this ELISA, an anti-CD40 monoclonal antibody is used as the capture antibody, is incubated with vector (control); GM-CSF; or sCD40/GM-CSF, followed by incubating with a biotin-labeled GM-CSF antibody for detection of the fusion protein. 3T3 cells transfected with either sCD40 or GM-CSF are negative in this assay, while 3T3 cells transfected with sCD40/GM-CSF fusion construct are positive. C. ELISA to detect GM-CSF expression by the fusion protein construct. 3T3 cells are transfected with the control vector or the sCD40/GM-CSF construct. A pair of GM-CSF-specific antibodies are used for capture and detection in this assay.

In a preferred embodiment of the present invention, tumor cells expressing a soluble form of CD40, optionally in combination with certain cytokines, confer long term specific systemic immunity to individuals receiving such cells. In embodiments in which the cells are tumor cells, autologous cells are those which are derived from the subject and which have majorhistocompatability (MHC) components such that such a tumor cell obtained from a different subject would be quickly rejected. Alternatively, in other embodiments, sCD40 may be expressed from cells lacking MHC class I and class II epitopes, whereby such a line may be administered without rejection to different individual subjects. Such an approach has the advantage of not requiring biopsy and culture of a subject's own tumor cells by standard procedures.

In another embodiment, the present invention is useful in the treatment or prevention of particular diseases through the choice of antigen optionally co-administered, and through the choice of cytokine or cytokines also optionally co-administered, where these optional components are co-administered with a means for supplying to the subject a soluble form of a CD40. Thus, where an antigen for a condition is known, it is within the scope of the present invention to co-administer a purified antigen optionally conjugated to a suitable hapten together with sCD40, or nucleic acid directing sCD40 expression in vivo, to a subject in order to elicit a systemic immune response to the antigen.

In other embodiments, a means for supplying a cytokine is provided in addition to a means for supplying a sCD40. Such means include means specified above for delivery of sCD40, including purified cytokine and nucleic acids directing production of same in vivo. By the term “cytokine” is meant the general class of hormones of the cells of the immune system, including cytokines, lymphokines, and others. Specific non-limiting examples include interleukins 1-12 (IL1 to IL-12), GM-CSF, M-CSF, LIF, LT, TGF-beta, gamma-interferon, TNF-alpha, BCGF, CD2 or ICAM. See, “Cytokines and Cytokine Receptors,” A. S. Hamblin, 1993, (D. Male, ed., Oxford University Press, New York). Specific embodiments provide for means whereby IL-12 and/or GM-CSF are simultaneously or sequentially provided to the individual. In certain embodiments, the cytokine and sCD40 are expressed from a transgene as a fusion protein.

In other embodiments of the invention, co-administration of an antigen is not required. For example, a soluble form of CD40 can be administered to an individual alone or in combination with a cytokine or cytokines, in order to induce a systemic immune response to, for example, a pre-existent antigen such as a poorly immunogenic tumor Thus, in this embodiment, the sCD40 is optionally administered without co-administration of an antigen or cytokine.

Alternatively, sCD40 may be provided as a purified protein, optionally in combination with an antigen, which may also be in a purified state. Further specific embodiments provide for co-administration of a cytokine, also optionally provided in a purified state. In these embodiments, sCD40 may be considered an immune adjuvant for antigen-based immune therapies.

Thus, the present invention addresses a method of inducing a systemic immune response in vivo utilizing sCD40, particularly sCD40 gene therapy.

An advantage of using sCD40 gene therapy instead of native CD40 gene therapy for inducing anti-tumor immune responses in vivo is that a soluble protein can reach more effector cells than a membrane bound protein.

The instant application includes a demonstration that transgenic expression of a sCD40 protein in vivo results in T cell activation, including production of Th1 cytokines, and stimulation of cytolytic activities of both cytotoxic T lymphocytes and natural killer cells. Significantly, local gene therapy with the sCD40 at the tumor site results in growth inhibition and regression of established tumors, including B16 melanoma. Importantly, while GM-CSF-cDNA based cancer vaccine, which is considered to be the most potent cancer vaccine, has little effect on the growth of tumors in several established tumor models, expression of the sCD40 by methods of the current invention, in combination with GM-CSF, induces potent growth inhibition of these established tumors. It is thus disclosed herein that a sCD40 protein can activate both T and NK cells in vivo, stimulating potent therapeutic immune responses, including but not limited to anti-tumor responses.

The following examples serve to more fully describe the manner of making and using the above-described invention, as well as to set forth various aspects of the invention. It is understood that these examples in no way serve to limit the true scope of this invention, but rather are presented for illustrative purposes.

### EXAMPLES

Material and Methods

Construction of sCD40 Vector

The CDNA encoding the murine membrane-bound CD40 is obtained by reverse transcriptase-polymerase chain reaction (RT-PCR) of total RNA prepared from spleens of Balb/c mice. Extraction of RNA and RT-PCR is performed as described. A pair of primers is synthesized according to the published sequences and used for amplication of mCD40. The forward primer, 5′-GTC GCT AGC GGG CAG TGT GTT ACG TGC AGT, corresponds to nucleotides 69-89, published in the Journal of Immunology vol 148, 620-626(2) 1992, which corresponds to a site starting immediately after the putative signal peptide of the mature murine CD40 protein. This primer includes the addition of a 5′ Nhel restriction enzyme site and a GTC sequence, the GTC allowing more efficient digestion of the Nhel site. The reverse primer, 5′-CTT GCT AGC ACA GAT GAC ATT AGT CTG ACT, corresponds to nucleotides 546-566 of the gene sequence published in the Journal of Immunology vol. 148, 620-626(2) 1992, which corresponds to a site starting immediately before the transmembrane domain of the mature murine CD40 protein. This primer includes the addition of a 5′ Nhel restriction enzyme site and a CTT sequence, the CTT allowing more efficient digestion of the Nhel site. The final gene product encodes only the extracellular portion of the mature peptide, and excludes the signal peptide, transmembrane and cytoplasmic domains. (FIG. 2)

Amplification of the mCD40 cDNA is performed and the PCR products are purified on a 1.5% agarose gel and directly cloned into the expression vector p at the NheI sites. The resulting construct, is fully sequenced and no mismatch to published sequence is found. To construct the sCD40 expression vector, another pair of primers is synthesized. The forward primer, 5′-GGGCAGTGTTACGTGCAGT-3′, corresponds to nucleotides 71-90, including a site at the beginning of the primer. Nucleotides 9-70 are predicted to encode the leader sequence. The reverse primer, 5′-ACAGATGACATTAGTCTGACT-3′, corresponds to nucleotides 545-566. The resulting CD40 cDNA portion (71-566) encoding the entire extracellular domain without the leader sequence is cloned into an expression vector. (FIG. 1)

Transfection of sCD40 Construct into Cell Lines

Mouse 3T3 fibroblasts are co-transfected with psCD40 and pSV₂-neo and selected in medium supplemented with 0.6 mg/ml G418. Expression of sCD40 by sCD40-3T3 stable subclones is confirmed by a CD40-specific Western blot using a rabbit polyclonal antibody against murine CD40.

NK Cell Cytolytic Assay

The effect of sCD40 on NK cytolytic activity in both tumor-bearing and tumor-free mice is determined. MPC myeloma tumor-bearing Balb/C mice are injected intramuscularly and intratumorally with 2 μg of plasmid DNA/injection encoding sCD40 or luciferase (control), using a cationic lipid transfection reagent, GenePortor. Plasmid DNA is injected on days 4 and 8 post tumor challenge. NK assay is performed on day 11. NK cytolytic assays are also performed in naïve Balb/C mice that had received skin transfection of plasmid vectors (12 μg of DNA each transfection). Skin transfection is performed via a gene gun. NK assay after gene gun transfection is performed 3 d later. Preparation of single-cell suspensions of splenocytes and cytolytic assays using ⁵¹Cr-labeled YAC-1 target cells are performed. (FIG. 3)

CTL Activity Assay

Mice are immunized by s.c. injection of tumor vaccines. Vaccines are prepared, for example, by (1) sCD40-3T3 cells are mixed with MPC11 tumor cells (1:1 ratio, 10⁶ cell each) and γ-irradiated (40 Gy); or (2) MPC11 tumor cells are transfected with sCD40 plasmid DNA via a gene gun, followed by γ-irradiation.

Results

Expression of a Membrane Bound CD40 Only Slightly Increases the Immunogenicity of MethA and B16 Tumors.

To determine the capacity of CD40 in stimulating anti-tumor responses, a CD40 expression vector encoding the entire CD40 protein is transfected into both Meth A and B16 tumors cells. The transfected cells are selected in G418 for neomycin expression. Based on flow cytometry analysis, over 90% of the transfected cells express CD40 on their cell surface. A single dose of 1×10⁵ neo-MethA (control or CD40-MethA cells is injected subcutaneously. While all the mice receiving neo-MethA tumor cells develop tumors within two weeks, none of the mice injected with CD40-MethA tumor cells develop palpable tumors. At a higher dose of injected cells, 2×10⁵ CD40-MethA cells causes tumor development in all of the mice. In addition, mice receiving CD40-B16 tumor cells all develop tumors, at a rate only slightly slower than neo-B16 injected control mice.

In vivo Transgenic Expression of a soluble CD40 (sCD40) results in regression of Established Murine Tumors

Offered as a possible explanation of the finding, but not so as to limit the invention, the lack of CD40 effect against poorly immunogenic tumors in vivo may be due to poor accessibility of CD40 to T cells. This is supported by the efficacy of secreted cytokines in activating T cells and NK cells.

The current invention shows that the expression of soluble CD40 protein in vivo results in better anti-tumor effect, using an expression vector encoding only the extracellular portion of CD40 (FIG. 1). The ability of the sCD40 vector to express sCD40 protein is determined by ELISA and Western blot.

The current invention shows that the sCD40 vector can be used as a cancer therapeutic agent, utilizing mice with established murine tumors and treating them with either direct skin transfection of sCD40 expression vector, psCD40, or irradiated tumor cells transfected with psCD40. In B16 tumor model, mice with 2-3 mm tumors received direct peritumoral and intramuscular transfection of either control vector or sCD40 vector. While the tumors in untreated mice and mice treated with luciferase expression vector grow progressively, a significant percent of B16 tumors in sCD40 vector treated mice completely regress (FIG. 7).

Soluble CD40-based Cancer Vaccine Induces Therapeutic Anti-tumor immunity and enhances the efficacy of GM-CSF-based Cancer Vaccine

A cancer vaccine composed of irradiated tumor cells transfected with sCD40 vector can induce tumor regression. Meth A tumor-bearing mice are treated with irradiated Meth A tumor cells transfected with either GM-CSF or sCD40 cDNA expression vectors. Whereas GM-CSF tumor vaccine has no effect on the growth of established Meth A tumors, 2 out of 4 tumors in mice receiving sCD40 tumor vaccines undergo complete regression. (FIG. 10)

To ensure that the absence of therapeutic effect of GM-CSF vaccine is not due to lack of GM-CSF expression, GM-CSF ELISA is performed with supernatant collected from GM-CSF-transfected Meth A cells. A high level of GM-CSF production (60-170 ng/ml/10⁶ cells) is detected in GM-CSF-transfected Meth A cells.

In vivo Expression of sCD40 Induces Th1 Cytokine Production and Cytolytic Activities of Both CTL and NK Cells

The inventors show the immunogenic potential of sCD40, by determining whether sCD40 could stimulate systemic cytokine production in vivo. Skin transfection of psCD40 is performed via a gene gun. Our results show that transgenic expression of sCD40 in vivo is capable of stimulating systemic production of Th1 cytokines, as elevated levels of both IFN-γ and IL-2 in plasma cells are detected. (FIG. 5B)

To further explore the immunogenic potential of sCD40, a CTL assay is performed with spleen cells from mice treated with irradiated cells transfected with and without sCD40. As shown in FIG. 5A, a vigorous tumor cell-specific CTL response in vivo is induced in mice treated with irradiated tumor cells transfected with sCD40. In contrast, little CTL activity against the tumor cells is detectable in mice treated with irradiated tumor cells alone.

NK cells are also known to express CD40 ligand (CD40L), it is therefore investigated whether transgenic expression of sCD40 could also activate NK cells. Naïve mice are given skin injection (via a gene gun) of either the sCD40 vector or the control vector (luceferase), PBLs are prepared from these treated mice 5 days later. A potent NK cytolytic activity is induced in mice that had received sCD40 transfection.

### FURTHER EXAMPLES

Thus, the interaction of CD40 and its ligand, CD154 (CD40L) plays a key role in the induction of cellular immune responses. Triggering CD40 receptor expressed on antigen-presenting cells (APC) results in activation of APC, which stimulates CD8+ T cells. Direct activation of APC either by using the CD40L or anti-CD40 antibody results in anti-tumor and antimetastatic effects involving both CD8+ T cell and NK cells. In the transgenic model of tumor-induced antigen-specific CD4+ T cells and production of Th1 cytokines in response to in vivo priming, resulting in conversion of T cell tolerance to T cell priming. Thus, as disclosed herein, sCD40 gene therapy directly activates tumor specific CD4+ T cells and results in anti-tumor and anti-tolerance effects in tumor-bearing hosts.

Evocation of T Cell Mediated Anti-tumor Response Using CD40

An expression vector encoding a soluble form of CD40 (sCD40) is constructed. The soluble form over a membrane bound form is preferable, mainly due to its high delivery efficiency. Expression of cytokines (which are soluble) can be relative high when delivered by a gene gun both in vivo or ex vivo. In addition, a soluble protein can also reach far more cells than a membrane bound one. As shown by Table 1, below, sCD40 gene therapy is capable of inducing regression of the murine Meth A tumor. (FIG. 10)

However, sCD40 alone has only limited inhibitory effects in the J558 tumor model and in the poorly immunogenic B16 tumor model (data not shown). Interestingly, while GM-CSF/IL-12 gene therapy fails to inhibit the growth of established J558 tumors, GM-CSF/IL-12 in combination with sCD40 gene therapy results in 100% regression of tumors. (FIG. 8)

The potential of sCD40 gene therapy in the poorly immunogenic MOPC and B16 tumor models is also tested. Results indicate that sCD40 can also greatly potentiate the anti-tumor effects mediated by GM-CSF/IL-12 vaccines. (FIGS. 6 and 9)

TABLE 1
Soluble CD40 gene therapy induces anti-tumor effects.

| | Meth A | J558 | MOPC |
|---|---|---|---|
| Vaccine | % tumor | % tumor | % tumor |
| Treatment | regression | regression | regression |
| Control* | 0 | 0 | 0 |
| GM-CSF | 0 | — | — |
| SCD40 | 67 | — | — |
| GM/IL12 | — | 0 | 0 |
| GM/sCD40/IL12 | — | 100 | 50 |

The conditions set forth in Table 1 are as follows: Control equals either naïve mice or mice received cell vaccine without gene transfection. Mice with s. c. Meth A tumors (4-5 mm in diameter) are immunized twice with irradiated Meth A tumor cells admixed with 3T3 cells transfected with either nothing, or GM-CSF (GM) cDNA or sCD40 cDNA. n=3 for each group. Seven days after implanting 1×10⁶ J558 tumor cells, mice receive irradiated J558 cells admixed with 3T3 cells transfected with either GM cDNA, or GM/sCD40, followed by an IL12 J558 vaccine. n=4. MOPC tumors (3 mm in diameter) are treated with either GM or GM plus sCD40 transfected MOPC vaccines, followed by an IL12 vaccine. n=4 for each group.

Activation of T Cells using sCD40 in Vivo

T-cell receptor (TCR) transgenic mice are used to maximize the detection of CD4+ T cell activation. Transgenic mice expressing an αβ T-cell receptor specific for a MHC class II epitope of influenza hemagglutinin (HA) are described, for example, by Sotomayor et al., Conversion of tumor-specific CD4+ T-cell tolerance to T-cell priming through in vivo ligation of CD40, Nature Med. 5:780-787 (1999), and can be obtained, for example, from H. Lee Moffitt Cancer Center. Antigen-specific CD4+ T cells transferred to mice bearing HA-expressing tumor cells become unresponsive to HA antigen. A stimulatory anti-CD40 antibody is capable of activating APC which in turn activates CD4+ T cells as evidenced by the expansion of HA-specific CD4+ T cells, and production of IL-2 and IFN-γ upon antigen restimulation ex vivo. Renca and A20 cells expressing HA (Renca^{HA}, A20^{HA}) are used.

The ability of sCD40 to stimulate CD4+ T cells in tumor free and tumor-bearing mice is investigated. Seven days after i.v. injection of 1×10⁶ Renca^{HA} cells, adoptive transfer of transgenic CD4+, anti-HA TCR+ T cells is performed as described by Sotomayor et al. Single-cell suspensions prepared from peripheral lymph nodes and spleens of TCR transgenic donors are tested for CD4 and clonotypic TCR by flow cytometry. A total of 2.5×10⁶ CD4+anti-HA TCR+ T cells are injected into the tail veins of tumor free and tumor-bearing mice. Ten days after transfer of T cells, at which time antigen-specific T-cell tolerance to the tumor in the tumor-bearing mice is established, the mice are treated with irradiated Renca^{HA} tumor cells transfected with either an empty vector or the sCD40 vector. Analysis of CD4+ clonal expansion and Th1 cytokine production is performed 10 days later as described. As an alternative approach, the sCD40 vector is also transfected intradermally via a gene gun, such as described in Tan, et al. IL-12 cDNA skin transfection potentiates human papillomavirus E6 DNA vaccine-induced anti-tumor immune response, Cancer Gene Therapy. (6) 4:331-339(1999).

Detection of CD4+ T cell expansion and production of Th1 cytokines in sCD40 vaccinated mice is indicative of direct activation of CD4+ T cells by sCD40 in vivo.

Experiments in transgenic mice encoding a T cell receptor specific for OVA (C57/BL background are also performed, see for example, Pape et al., Direct evidence that functionally impaired CD4+ T cells persist in vivo following induction of peripheral tolerance, 160 (10):4719-29 (1998).

sCD40/GM-SCF Fusion Protein Plasmid Vaccine Gene Therapy

Although GM-CSF/IL-12 tumor cell vaccines fail to inhibit the growth of established B16 tumors, sCD40 and GM-CSF/IL-12 combinational cancer vaccines exhibit significant anti-tumor effect against the established B16 tumors. It is determined whether IL-12 or GM-CSF, plays the critical role in providing sCD40 the extra anti-tumor activity. For ease of transfection and for a possible better effect, a construct encoding a sCD40-GM-CSF fusion protein is made. (FIG. 13)

A fusion protein often works better than when the proteins are separate, possibly due to the closer proximity of the proteins. Thus, a vector encoding sCD40-IL-12 fusion protein is also made. The efficacy of a vaccine encoding sCD40-GM-CSF fusion protein compared to GM-CSF-IL-12 vaccine and GM-CSF vaccine is investigated. The sCD40-GM-CSF vaccine causes significant tumor regression as shown in FIG. 11.

Each of these two fusion protein-encoding vectors are tested, in comparison with sCD40, GM-CSF and IL-12, to determine which fusion vector activates CD4+ T cells more powerfully. Immunization with the most powerful fusion protein vector is also compared to sCD40/GM-CSF/IL-12 vaccine. Whether sCD40/cytokine fusion protein treatment followed by another cytokine will further activate T cells is also determined. Sequential immunizations with sCD40/GM-CSF followed a week later by IL-12 immunization generate substantially more anti-tumor effect than simultaneous immunization with the two cytokines.

In addition to examining the effect of sCD40/cytokine on CD4+ T cells, their effect on NK cells, which have been shown to play an important role in cancer immunotherapy, is also determined. Like CD4+ T cells, NK cells express CD40L. Indirect activation of NK cells by a stimulatory anti-CD40 antibody results in potent anti-tumor and anti-metastasis activity. Depletion of NK cells in vivo either reduces significantly or abrogates the anti-tumor effect mediated by the anti-CD40 antibody. Whether sCD40 gene therapy can augment NK cytolytic activity against YAC-1 target cells in mice receiving vectors encoding various cytokines, sCD40 and their fusion proteins is also determined. Identification of the most effective sCD40/cytokine combination to activate CD4+ T cells and NK cells is determined. To evaluate the therapeutic anti-tumor efficacy of sCD40/cytokine fusion protein, the genetic immunotherapy in two tumor models, the subcutaneous B16 tumor model and the highly metastatic NXS2 neuroblastoma model are tested. Both tumor models are poorly immunogenic, and NXS2 are also known to be highly sensitive to NK cells. For B16 tumor model, 1×10⁵ cells is used to challenge mice. After about 10 days when tumor tolerance is established, genetic cancer vaccine with sCD40/cytokine or sCD40/cytokine fusion protein is performed. The optimal combination of sCD40/cytokine(s) and the timing of immunization is used that generates the highest levels of CD4+/NK cell activation. To evaluate the anti-metastatic effect of sCD40/cytokine, 1×10⁵ NXS2 neuroblastoma cells are injected i.v. into A/J mice to induce live metastasis. Five to 7 days after tumor implantation, sCD40/cytokine-based tumor cell vaccine are administered. Liver metastases are evaluated as described, for example, by Lode et al., Natural killer cell-mediated eradication of neuroblastoma metastases to bone marrow by targeted interleukin-2 therapy, Blood. 91:1706-1715 (1998).

To determine the functional participation of immune cells in mediating anti-tumor/anti-metastasis effects as a result of sCD40/cytokine tumor cell vaccination, in vivo depletion of CD4+, CD8+ and NK cells are performed. The requirement of CD40/CD40L ligation for induction of anti-tumor effects in vivo is confirmed by using CD40L knockout mice (provided by Dr. Flavel, Yale University). Lack of sCD40-mediated anti-tumor effect in the CD40L deficient mice suggests that sCD40 induces anti-tumor effects via CD40L.

Thus it is shown that transgenic expression of a soluble form of CD40 (sCD40) produces therapeutic anti-tumor effects, for example, in murine tumor models. It is also shown that transgenic expression of sCD40 can greatly enhance the efficacy of GM-CSF tumor vaccine in inducing growth inhibition of established murine tumors. Therefore, sCD40 serves as a potent anti-cancer immunotherapeutic agent.

All of the above-cited patents, publications, and references are hereby expressly incorporated by way of reference in their respective entireties.

It should be apparent to one of ordinary skill in the art that other embodiments can be readily contemplated in view of the teachings of the present specification. Such other embodiments, while not specifically disclosed nonetheless fall within the scope and spirit of the present invention. Thus, the present invention should not be construed as being limited to the specific embodiments described above, and is solely defined by the following claims.

What is claimed is:

1. A method of stimulating a systemic immune response in a subject in need thereof, comprising administering to the subject a therapeutically effective amount of cells, wherein said cells are proliferation incompetent and have been genetically engineered to express sCD40.

2. The method of claim 1 wherein said cells are autologous or heterologous to said subject.

3. The method of claim 1 wherein said subject is a human.

4. The method of claim 1 wherein said tumor cells are rendered proliferation incompetent by gamma irradiation.

5. The method of claim 1 wherein said cells are tumor cells, bone marrow cells, stem cells, fibroblasts, lymphocytes, or combinations thereof.

6. The method of claim 1 further comprising administration of one or more cytokines.

7. The method of claim 6 in which said cytokine is granulocyte-macrophage colony stimulating factor, IL-12 or both.

8. The method of claim 6 in which said cytokine is granulocyte-macrophage colony stimulating factor and said cytokine and sCD40 are expressed separately or as a fusion protein.

9. The method of claim 1 in which said cells are administered systemically, peritoneally, intramuscularly, or intradermally.

10. A composition comprising a cell expressing a fusion protein comprising sCD40 linked to granulocyte-macrophage colony stimulating factor.

11. A method of stimulating a systemic immune response in a subject having an established tumor, comprising administering to the subject a therapeutically effective amount of a recombinant nucleic acid through which a protein comprising sCD40 can be expressed.

12. A method of stimulating a systemic immune response in a subject in need thereof, comprising administering to the subject a therapeutically effective amount of sCD40.

13. The method of claim 13 further comprising the step of co-administering to said subject an antigen, whereby a systemic immune response is induced to said antigen.

14. A method of suppressing growth of a tumor in a subject, comprising providing to said subject a therapeutically effective amount of sCD40 in vivo such that growth of a tumor in said subject is suppressed.

15. A method of stimulating a systemic immune response in a subject in need thereof, comprising administering to the subject a therapeutically effective amount of cells, wherein said cells are characterized by: (s) a substantial lack of expression of MHC class I and class II molecules, and (b) an ability to express sCD40.
